SEQUENCE LISTING (3 sequences)

SEQ ID NO: 1 - DNA, 20 bp, synthetic construct
  source 1..20: mol_type = other DNA
agagtttgatcctggctcag

SEQ ID NO: 2 - DNA, 19 bp, synthetic construct
  source 1..19: mol_type = other DNA
ggttaccttgttacgactt

SEQ ID NO: 3 - DNA, 1159 bp, Lactobacillus johnsonii
  source 1..1159: mol_type = genomic DNA
cccattctcttgtctaccttagacggctgactcctataaaggttatcccaccggctttgggtgttacagactctcatggtgtgacgggcggtgtgtacaaggcccgggaacgtattcaccgcggcgtgctgatccgcgattactagcgattccagcttcgtgtaggcgagttgcagcctacagtccgaactgagaacggctttaagagatccgcttgccttcgcaggttcgcttctcgttgtaccgtccattgtagcacgtgtgtagcccaggtcataaggggcatgatgacttgacgtcatccccaccttcctccggtttgtcaccggcagtctcattagagtgcccaacttaatgatggcaactaatgacaagggttgcgctcgttgcgggacttaacccaacatctcacgacacgagctgacgacagccatgcaccacctgtctcagcgtccccgaagggaacacctaatctcttaggtttgcactggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcagcactgagaggcggaaacctcccaacacttagcactcatcgtttacggcatggactaccagggtatctaatcctgttcgctacccatgctttcgagcctcagcgtcagttgcagaccagagagccgccttcgccactggtgttcttccatatatctacgcattccaccgctacacatggagttccactctcctcttctgcactcaagttcaacagtttctgatgcaattctccggttgagccgaaggctttcacatcagacttattgaaccgcctgcactcgctttacgcccaataaatccggacaacgcttgccacctacgtattaccgcggctgctggcacgtagttagccgtgactttctaagtaattaccgtcaaataaaggccagttactacctctatctttcttcactaccacagagctttacgagccgaaacccttcttcactccacgcggcgtgctcatcgactttcgtccatgtgagtcatcgtcgtctctctcgctctcgcacaaaaacaaaaaaaa

(19) United States
(12) Patent Application Publication
(10) Pub. No.: US 2024/0016863 A1
(43) Pub. Date: Jan. 18, 2024

(54) LACTOBACILLUS JOHNSONII AND METHOD FOR INCREASING THE LEVEL OF UNSATURATION IN FATTY ACID USING LACTOBACILLUS JOHNSONII OR METABOLITES THEREOF

(71) Applicant: TCI CO., LTD., Taipei (TW)

(72) Inventors: YUNG-HSIANG LIN, Taipei (TW); CHU-HAN HUANG, Taipei (TW); YI-LIN CHEN, Taipei (TW); CHIH-I LIAO, Taipei (TW)

(21) Appl. No.: 18/349,986

(22) Filed: Jul. 11, 2023

Related U.S. Application Data
(60) Provisional application No. 63/359,885, filed on Jul. 11, 2022.

(51) Int. Cl. A61K 35/747; C12N 1/20; A61P 3/06; A61Q 17/04; A61K 8/99

(52) U.S. Cl. CPC A61K 35/747; C12N 1/205; A61P 3/06; A61Q 17/04; A61K 8/99; C12R 2001/225

(57) ABSTRACT

Lactobacillus johnsonii is provided. The Lactobacillus johnsonii is Lactobacillus johnsonii TCI369 with an accession number of DSM 34008. Also, a method for increasing the level of unsaturation in fatty acids is provided. The method includes administering to a subject in need thereof a composition including Lactobacillus johnsonii or metabolites thereof. The Lactobacillus johnsonii is the Lactobacillus johnsonii TCI369 with an accession number of DSM 34008.

### CROSS-REFERENCE TO RELATED APPLICATION

This application claims the benefit of U.S. provisional application Ser. No. 63/359,885, filed on Jul. 11, 2022. The entirety of the above-mentioned patent application is hereby incorporated by reference herein and made a part of the specification.

### REFERENCE OF AN ELECTRONIC SEQUENCE LISTING

The contents of the electronic sequence listing (P223423USI.xml; Size: 5 KB; and Date of Creation: Jul. 11, 2023) is herein incorporated by reference in its entirety.

### BACKGROUND

### Technical Field

The present disclosure relates to Lactobacillus johnsonii, and particularly relates to Lactobacillus johnsonii and a method for increasing the level of unsaturation in fatty acids using Lactobacillus johnsonii or metabolites thereof.

### Related Art

Fatty acid can be divided into “saturated fatty acids” and “unsaturated fatty acids”. Saturated fatty acids easily cause cardiovascular diseases, and essential fatty acids for a human body belong to unsaturated fatty acids, such as Omega-3 or Omega-6 fatty acid.

In order to solve the problems above, it is urgent for those skilled in the art to develop probiotic products with scientific basis and high efficiency to benefit the vast population in need thereof.

### SUMMARY

In view of this, the present disclosure provides Lactobacillus johnsonii and a method for increasing the level of unsaturation in fatty acids using the Lactobacillus johnsonii or metabolites thereof.

In some embodiments, Lactobacillus johnsonii is provided, and the Lactobacillus johnsonii is Lactobacillus johnsonii TCI369 with an accession number of DSM 34008.

In some embodiments, a use of Lactobacillus johnsonii or metabolites thereof in preparing a composition for increasing the level of unsaturation in fatty acids is provided, and the Lactobacillus johnsonii is Lactobacillus johnsonii TCI369 with an accession number of DSM 34008.

In some embodiments, a method for increasing the level of unsaturation in fatty acids is provided, including administering to a subject in need thereof a composition including Lactobacillus johnsonii or metabolites thereof. The Lactobacillus johnsonii is Lactobacillus johnsonii TCI369 with an accession number of DSM 34008.

In some embodiments, the Lactobacillus johnsonii is used for decomposing fat or metabolizing cholesterol of the subject.

In some embodiments, the Lactobacillus johnsonii is used for reducing triglycerides of the subject.

In some embodiments, the Lactobacillus johnsonii is used for reducing low-density lipoproteins or increasing high-density lipoproteins of the subject.

In some embodiments, the Lactobacillus johnsonii is used for regulating the balance of intestinal microbiota of the subject.

In some embodiments, the Lactobacillus johnsonii is used for strengthening the intestinal barrier of the subject.

In some embodiments, the Lactobacillus johnsonii is used for resisting ultraviolet light for the subject.

In some embodiments, the Lactobacillus johnsonii is used for protecting eyes of the subject.

In conclusion, the Lactobacillus johnsonii according to any embodiment can increase the level of unsaturation in fatty acids. In some embodiments, the Lactobacillus johnsonii or metabolites thereof according to any embodiment are suitable for preparing the composition for increasing the level of unsaturation in fatty acids. In some embodiments, the method for increasing the level of unsaturation in fatty acids includes: administering to the subject in need thereof the composition including the Lactobacillus johnsonii or metabolites thereof according to any embodiment. In some embodiments, the composition has the function of increasing the level of unsaturation in fatty acids. In some embodiments, the Lactobacillus johnsonii, the metabolites thereof or the prepared composition thereof also has one or more functions of decomposing fat, metabolizing cholesterol, reducing triglycerides, reducing low-density lipoproteins, increasing high-density lipoproteins, regulating the balance of intestinal microbiota, strengthening the intestinal barrier, resisting ultraviolet light and protecting eyes. In some embodiments, the method for decomposing fat, metabolizing cholesterol, reducing triglycerides, reducing low-density lipoproteins, increasing high-density lipoproteins, regulating the balance of intestinal microbiota, strengthening the intestinal barrier, resisting ultraviolet light and/or protecting eyes includes: administering to the subject in need thereof the Lactobacillus johnsonii, the metabolites thereof or the prepared composition thereof.

### BRIEF DESCRIPTION OF THE DRAWINGS

FIG. 1 is a bar chart showing the iodine value of oil after treated by a Lactobacillus johnsonii TCI369 sample in accordance with some embodiments of the present invention

FIG. 2 is a bar chart showing the relative cholesterol uptake in hepatocytes after treated by a Lactobacillus johnsonii TCI369 sample in accordance with some embodiments of the present invention.

FIG. 3 is a bar chart showing the relative glycerol secretion content of cells after treated by a Lactobacillus johnsonii TCI369 sample in accordance with some embodiments of the present invention.

FIG. 4 is a bar chart showing the relative viability of cells after treated by a Lactobacillus johnsonii TCI369 sample in accordance with some embodiments of the present invention.

FIG. 5 is a bar chart showing the concentration of triglycerides in blood in human subjects at week 0, week 2 and week 4 after ingesting a composition including Lactobacillus johnsonii TCI369 in accordance with some embodiments of the present invention.

FIG. 6 is a bar chart showing the concentration of high-density lipoproteins in human subjects at week 0, week 2 and week 4 after ingesting a composition including Lactobacillus johnsonii TCI369 in accordance with some embodiments of the present invention.

FIG. 7 is a bar chart showing the concentration of very low-density lipoproteins in human subjects at week 0, week 2 and week 4 after ingesting a composition including Lactobacillus johnsonii TCI369 in accordance with some embodiments of the present invention.

FIG. 8 is a bar chart showing the mean difference in features of intestinal microbiota in human subjects at week 0 and week 4 after ingesting a composition including Lactobacillus johnsonii TCI369 in accordance with some embodiments of the present invention.

FIG. 9 is a bar chart showing the relative gene expression of gut-associated pathogenic bacteria proteins in human subjects at week 0 and week 4 after ingesting a composition including Lactobacillus johnsonii TCI369 in accordance with some embodiments of the present invention.

### DETAILED DESCRIPTION

Herein, when referring to contents, the content unit “%” generally refers to weight percentage.

In some embodiments, Lactobacillus johnsonii is Lactobacillus johnsonii TCI369. The Lactobacillus johnsonii TCI369 is accessed in Food Industry Research and Development Institute (Taiwan) with an accession number of BCRC 911071, and is accessed in Deutsche Sammlung von Mikroorganismen and Zellkulturen (DSMZ, Germany) with an accession number of DSM 34008.

In some embodiments, the Lactobacillus johnsonii TCI369 is isolated from water in Wolongtan Pool, China.

In some embodiments, the Lactobacillus johnsonii or metabolites thereof have the capability of increasing the level of unsaturation in fatty acids. Therefore, the Lactobacillus johnsonii or metabolites thereof are suitable for preparing a composition for increasing the level of unsaturation in fatty acids.

In some embodiments, a method for increasing the level of unsaturation in fatty acids includes: administering to a subject in need thereof a composition including Lactobacillus johnsonii or metabolites thereof.

In some embodiments, the Lactobacillus johnsonii or metabolites thereof have the capability of decomposing fat. In other words, the Lactobacillus johnsonii or metabolites thereof can decompose the fat of a subject when being administered to the subject. Therefore, the Lactobacillus johnsonii or metabolites thereof are suitable for preparing a composition for decomposing fat.

In some embodiments, a method for decomposing fat includes: administering to a subject in need thereof a composition including Lactobacillus johnsonii or metabolites thereof.

In some embodiments, the Lactobacillus johnsonii or metabolites thereof have the capability of metabolizing cholesterol. In other words, the Lactobacillus johnsonii or metabolites thereof can metabolize cholesterol of a subject when being administered to the subject. Thus, the Lactobacillus johnsonii or metabolites thereof are suitable for preparing a composition for metabolizing cholesterol.

In some embodiments, a method for metabolizing cholesterol includes: administering to a subject in need thereof a composition including Lactobacillus johnsonii or metabolites thereof.

In some embodiments, the Lactobacillus johnsonii or metabolites thereof have the capability of reducing triglycerides. In other words, the Lactobacillus johnsonii or metabolites thereof can reduce triglycerides of a subject when being administered to the subject. Thus, the Lactobacillus johnsonii or metabolites thereof are suitable for preparing a composition for reducing triglycerides.

In some embodiments, a method for reducing triglycerides includes: administering to a subject in need thereof a composition including Lactobacillus johnsonii or metabolites thereof.

In some embodiments, the Lactobacillus johnsonii or metabolites thereof have the capability of reducing low-density lipoproteins. In other words, the Lactobacillus johnsonii or metabolites thereof can reduce the low-density lipoproteins of a subject when being administered to the subject. Therefore, the Lactobacillus johnsonii or metabolites thereof are suitable for preparing a composition for reducing the low-density lipoproteins.

In some embodiments, a method for reducing low-density lipoproteins includes: administrating a subject in need thereof a composition including Lactobacillus johnsonii or metabolites thereof.

In some embodiments, the Lactobacillus johnsonii or metabolites thereof have the capability of increasing high-density lipoproteins. In other words, the Lactobacillus johnsonii or metabolites thereof can increase the high-density lipoproteins of a subject when being administrated to the subject. Therefore, the Lactobacillus johnsonii or metabolites thereof are suitable for preparing a composition for increasing the high-density lipoproteins.

In some embodiments, a method for increasing high-density lipoproteins includes: administrating a subject in need thereof a composition including Lactobacillus johnsonii or metabolites thereof.

In some embodiments, the Lactobacillus johnsonii or metabolites thereof have the capability of regulating the balance of intestinal microbiota. In other words, the Lactobacillus johnsonii or metabolites thereof can regulate the balance of the intestinal microbiota of a subject when being administrated to the subject. Therefore, the Lactobacillus johnsonii or metabolites thereof are suitable for preparing a composition for regulating the balance of the intestinal microbiota.

In some embodiments, a method for regulating the balance of intestinal microbiota includes: administering to a subject in need thereof a composition including Lactobacillus johnsonii or metabolites thereof.

In some embodiments, the Lactobacillus johnsonii or metabolites thereof have the capability of strengthening the intestinal barrier. In other words, the Lactobacillus johnsonii or metabolites thereof can strengthen the intestinal barrier of a subject when being administered to the subject. Therefore, the Lactobacillus johnsonii or metabolites thereof are suitable for preparing a composition for strengthening the intestinal barrier.

In some embodiments, a method for strengthening the intestinal barrier includes: administering to a subject in need thereof a composition including Lactobacillus johnsonii or metabolites thereof.

In some embodiments, the Lactobacillus johnsonii or metabolites thereof have the capability of resisting ultraviolet light. In other words, the Lactobacillus johnsonii or metabolites thereof can resist the ultraviolet light for a subject when being administered to the subject. Therefore, the Lactobacillus johnsonii or metabolites thereof are suitable for preparing a composition for resisting the ultraviolet light.

In some embodiments, a method for resisting ultraviolet light includes: administering to a subject in need thereof a composition including Lactobacillus johnsonii or metabolites thereof.

In some embodiments, the Lactobacillus johnsonii or metabolites thereof have the capability of protecting eyes. In other words, the Lactobacillus johnsonii or metabolites thereof can protect the eyes of a subject when being administered to the subject. Therefore, the Lactobacillus johnsonii or metabolites thereof are suitable for preparing a composition for protecting the eyes.

In some embodiments, a method for protecting eyes includes: administering to a subject in need thereof a composition including Lactobacillus johnsonii or metabolites thereof.

In some embodiments, the Lactobacillus johnsonii is included in the composition in a form of bacterial powder.

In some embodiments, the Lactobacillus johnsonii is included in the composition in a form of viable bacteria or dead bacteria.

In some embodiments, an effective dosage of the Lactobacillus johnsonii is 100 mg/day.

In some embodiments, the subject may be a human.

In some embodiments, the composition may be a pharmaceutical composition or an edible composition for non-medical purposes.

In some embodiment, when the composition is a pharmaceutical composition, the pharmaceutical composition includes an effective dosage of the Lactobacillus johnsonii. The pharmaceutical composition can be manufactured into a form suitable for enteral, parenteral, oral or topical administration using technologies well-known by those skilled in the art.

In some embodiments, the form suitable for enteral or oral administration may be, but is not limited to, a tablet, a troche, a lozenge, a pill, a capsule, a dispersible powder, a granule, a solution, a suspension, an emulsion, a syrup, an elixir, a slurry or the like.

In some embodiments, the form suitable for parenteral or topical administration may be, but is not limited to, an injection (for example, a sterile aqueous solution or a dispersion), a sterile powder, an external preparation or the like.

In some embodiments, the dosage form of the injection may be, but is not limited to, intraperitoneal injection, subcutaneous injection, intraepidermal injection, intradermal injection, intramuscular injection, intravenous injection or intralesional injection.

In some embodiments, the pharmaceutical composition including an effective dosage of the Lactobacillus johnsonii may further include a pharmaceutically acceptable carrier widely used in a medicine manufacturing technology. In some embodiments, the pharmaceutically acceptable carrier can be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome or the like. The types and quantities of carriers used are within the professional and routine technical scope of those skilled in the art. The solvent for the pharmaceutically acceptable carrier may be water, normal saline, phosphate buffered saline (PBS) or an alcohol containing aqueous solution.

In some embodiments, the pharmaceutical composition including an effective dosage of the Lactobacillus johnsonii may be manufactured into an external preparations suitable for topically administering to skin by technologies well-known to those skilled in the art, including, but not limited to, an emulsion, a gel, an ointment, a cream, a patch, a liniment, a powder, an aerosol, a spray, a lotion, a serum, a paste, a foam, a drop, a suspension, a salve and a bandage.

In some embodiments, when the pharmaceutical composition is an external preparation, the pharmaceutical composition may be prepared by mixing an effective dosage of the Lactobacillus johnsonii and a base well-known to those skilled in the art.

In some embodiments, the base may include one or more of the following additives: water, alcohols, glycol, hydrocarbons (such as petroleum jelly and white petrolatum), wax (such as paraffin and yellow wax), preserving agents, antioxidants, surfactants, absorption enhancers, stabilizing agents, gelling agents (such as Carbopol® 974P, microcrystalline cellulose and carboxymethyl cellulose), active agents, humectants, odor absorbers, fragrances, pH adjusting agents, chelating agents, emulsifiers, occlusive agents, emollients, thickeners, solubilizing agents, penetration enhancers, anti-irritants, colorants, propellants and the like. The selection and quantity of these additives are within the professional and routine technical scope of those skilled in the art.

In some embodiments, when the composition is an edible composition for non-medical purposes, the edible composition includes an effective dosage of the Lactobacillus johnsonii. The edible composition may be in the form of powder, granules, solution, colloid or paste.

In some embodiments, the edible composition including the Lactobacillus johnsonii may be a food product or a food additive.

In some embodiments, the edible composition including the Lactobacillus johnsonii may be beverages, fermented foods, bakery products, health foods, dietary supplements, or the like. In some embodiments, the edible composition including the Lactobacillus johnsonii may further include an adjuvant. For example, the adjuvant may be maltodextrin, malic acid, sucralose, citric acid, fruit flavor, honey flavor, steviol glycoside or a combination thereof. The type and quantity of the selected adjuvant is within the professional and routine technical scope of those skilled in the art.

In some embodiments, the food additive may be seasonings, sweeteners, flavor, pH regulators, emulsifiers, colorant, stabilizers, or the like.

In the following examples, unless otherwise specified, the experiment steps are performed at room temperature (about 25° C.) and normal pressure (1 atm).

### Example 1: Strain Identification

An isolated strain isolated from water of Wolongtan Pool, China was subjected to strain identification. First, the 16S ribosomal gene (16S rRNA) of the isolated strain was transcribed into complementary DNA (cDNA), then a PCR product of the isolated strain was obtained by a 16S ribosomal gene primer pair (shown in Table 1) and polymerase chain reaction (PCR). Later, sequencing was carried out by Sanger sequencing to obtain a 16S ribosomal gene sequence (i.e., SEQ ID NO: 3). Subsequently, the sequence of SEQ ID NO:3 was aligned with 16S ribosomal gene sequences of other Lactobacillus johnsonii on the web site of the National Center of Biotechnology Information (NCBI), USA, to obtain a result that the identity (Per. Ident) between the 16S ribosomal gene sequence of the isolated strain and the 16S ribosomal gene sequences of the other Lactobacillus johnsonii was 99.37% to 99.46%, as shown in Table 2. Therefore, the isolated strain was named Lactobacillus johnsonii TCI369.

TABLE 1

| Name of | Sequence | |
|---|---|---|
| primer | number | Sequence |
| 8F | SEQ ID NO: 1 | 5′-AGAGTTTGATCCTGGCTCAG-3′ |
| 1492R | SEQ ID NO: 2 | 5′-GGTTACCTTGTTACGACTT-3′ |

TABLE 2

| | Percent |
|---|---|
| | Identity |
| Lactobacillus johnsonii | (Per. Ident) |
| Lactobacillus johnsonii strain 15QC3AN 16s ribosomal | 99.46% |
| RNA gene, partial sequence | |
| Lactobacillus johnsonii strain KLDS 1.0734 16s ribosomal | 99.46% |
| RNA gene, partial sequence | |
| Lactobacillus johnsonii strain KLDS 1.0731 16s ribosomal | 99.37% |
| RNA gene, partial sequence | |
| Lactobacillus johnsonii strain 1019 16s ribosomal RNA | 99.46% |
| gene, partial sequence | |
| Lactobacillus johnsonii strain LBJ-1297 16s ribosomal | 99.46% |
| RNA gene, partial sequence | |
| Lactobacillus johnsonii strain G2A chromosome, complete | 99.37% |
| genome | |
| Lactobacillus johnsonii strain DC22.2 chromosome, | 99.37% |
| complete genome | |
| Lactobacillus johnsonii ALB-7 gene for 16s ribosomal | 99.37% |
| RNA, partial sequence | |

### Example 2: Preservation and Cultivation Experiments of Lactobacillus johnsoniiTCI369

1. The Lactobacillus johnsonii TCI369 (SEQ ID NO:3) isolated in Example 1 was cultured in an MRSD medium (purchased from BD, product number: 288130) to obtain a bacterial solution, and then the bacterial solution was mixed with glycerol in a ratio of 4:1. Then the mixture of the bacterial solution and the glycerol was stored at −80° C.

2. The Lactobacillus johnsonii TCI369 was inoculated into the MRSD medium by a bacterium inoculation amount of 1% (v/v) (about 1×10⁴ CFU/mL), and cultured at 37° C. for 24 h to form a Lactobacillus johnsonii TCI369 bacterial solution.

3. The Lactobacillus johnsonii TCI369 bacterial solution was centrifuged for 5 min at 5,000 rpm to obtain a supernatant, and the supernatant was filtered by a 0.2 μm filter membrane to obtain a filtrate, i.e., a Lactobacillus johnsonii TCI369 sample (i.e., the Lactobacillus johnsonii TCI369 sample included metabolites of the Lactobacillus johnsonii TCI369).

### Example 3: The Level of Unsaturation in Fatty Acids Test

A. Materials

1. MRSD medium: Purchased from BD; product number: 288130.

2. Sunflower oil: Brand: The brand is Standard Foods; Product name: Great Day.

B. Test Process:

1. An MRSD medium containing 5 wt % of sunflower oil was used as a control group, and an MRSD medium containing 5 wt % of sunflower oil and 0.25% (v/v) of the Lactobacillus johnsonii TCI369 sample prepared in the Example 2 was used as experimental group. wasAfter each group was reacted at room temperature for 24 h, the iodine value (IV) of each group was detected by SGS.

C. Test Result:

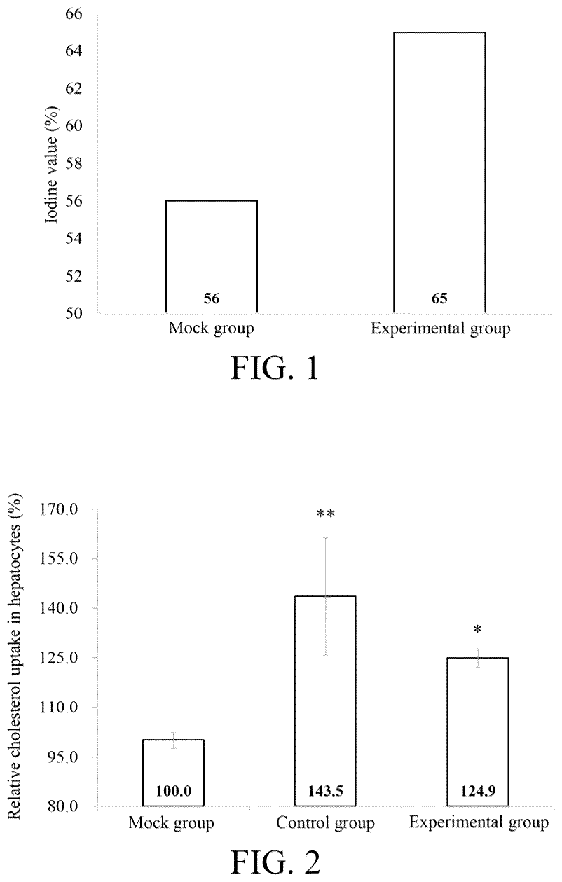

Refer to FIG. 1, the IV of the control group was 56%, and the IV of the experimental group was 65%. That is, compared with the control group, after the Lactobacillus johnsonii TCI369 sample was added to the experimental group, the IV of the experimental group was increased by about 16%.

Therefore, the Lactobacillus johnsonii TCI369 sample can increase the IV of the sunflower oil. The IV refers to the mass (g) of iodine absorbed by every 100 g of oil (or other samples). The larger the IV was, the larger the level of unsaturation in fatty acids was; otherwise, the smaller the IV was, the smaller the level of unsaturation in fatty acids was. In other words, it could be seen from the experimental results that the Lactobacillus johnsonii TCI369 and/or metabolites thereof have the effects of increasing the level of unsaturation in fatty acids and converting the fatty acids. The Lactobacillus johnsonii TCI369 and/or metabolites thereof can convert the ingested oil into unsaturated oil in vivo.

### Example 4: Cholesterol Metabolism Test

A. Materials and Instruments:

1. Cell line: Human liver cells, purchased from ATCC (American Type Culture Collection), cell number: HB-8065, hereinafter referred to as HepG2 cells.

2. Cell medium: DMEM (Dulbecco's modified Eagle's medium) (purchased from Gibco, product number: 12100-046); and 10% of fetal bovine serum (purchased from Gibco, product number: 10437-028) and 1% of penicillin-streptomycin (purchased from Gibco, product number: 15140122) were added.

3. Serum-free cell medium: DMEM (Dulbecco's modified Eagle's medium, purchased from Gibco, product number: 12100-046); and 1% of penicillin-streptomycin (purchased from Gibco, product number: 15140122) was added.

4. Trypsin: Diluted with 10× trypsin (purchased from Gibco, product number: 15400-054) and DPBS (Dulbecco's phosphate-buffered saline). The volume of DPBS is 9 times the volume of 10× trypsin.

5. Cholesterol Uptake Cell-based Assay Kit: purchased from Cayman; product number: 600440. This cholesterol uptake cell-based assay kit included U-18666A and NBD cholesterol.

6. Flow cytometer: purchased from BD Company.

B. Test Process:

1. The HepG2 cells were inoculated into a 6-well culture plate containing 2 mL of cell medium in each well according to a density of 1×10 5 cells per well, and cultured at 37° C. for 24 h. The HepG2 cells were divided into three test groups, i.e., a Mock group, a control group and an experiment group, respectively. Each group underwent trial in triplicate.

2. After culturing for 24 h, the medium of each group was replaced with an experiment medium, and each group was cultured at 37° C. for 24-72 h. The experiment medium of the Mock group was a serum-free cell medium containing 20 μg/mL of NBD cholesterol. The experiment medium of the control group was a serum-free cell medium containing 20 μg/mL of NBD cholesterol and 1.25 μM of U-18666A. The experiment medium of the experiment group was a serum-free cell medium containing 20 μg/mL of NBD cholesterol and 0.25% (v/v) of the Lactobacillus johnsonii TCI369 sample prepared in the Example 2.

3. After culturing for 24-72 h, the experiment medium of each group was removed after culturing, and rinsed with DPBS twice.

4. After rinsing, the cells of each group were processed according to the test process provided by the cholesterol uptake cell-based assay kit to obtain a test sample of each group; the parameters of the flow cytometer were set as excitation spectra and emission spectra of FITC, and then a green fluorescence signal of each group was detected by using the flow cytometer.

C. Test Result:

The relative cholesterol uptake in hepatocytes of all groups was calculated according to the following formula: relative cholesterol uptake in hepatocytes (%)=(green fluorescence signal of each group/green fluorescence signal of the Mock group)×100%.

The statistically significant difference between the test results of the Mock group and other groups is obtained by statistical analysis of student t-test. In the figure, “*” represents that the p value is less than 0.05 as compared with the Mock group, “*” represents that the p value is less than 0.01 as compared with the Mock group, and “***” represents that the p value is less than 0.001 as compared with the Mock group.

Refer to FIG. 2, the cells in the Mock group are only provided with cholesterol for processing, so the test result of the Mock group represents the performance of the cells under a normal physiological metabolism condition. Here, in a case where a relative cholesterol uptake in hepatocytes of the Mock group was set as 100%, a relative cholesterol uptake in hepatocytes of the control group was 143.5%, and a relative cholesterol uptake in hepatocytes of the experimental group was 124.9%. That is, compared with the Mock group, the relative cholesterol uptake in hepatocytes of the control group was significantly increased by about 43.5% after the cells in the control group were provided with cholesterol and U-18666A. Compared with the Mock group, the relative cholesterol uptake in hepatocytes of the experimental group was significantly increased by about 24.9% after the cells in the experimental group were provided with cholesterol and the Lactobacillus johnsonii TCI369 sample.

Therefore, the Lactobacillus johnsonii TCI369 sample can significantly improve the cholesterol uptake by hepatocytes. In other words, it could be seen from the experimental results that the Lactobacillus johnsonii TCI369 and/or metabolites thereof have the effects of significantly improving the cholesterol uptake by hepatocytes and promoting cholesterol metabolism. The Lactobacillus johnsonii TCI369 and/or metabolites thereof can promote cholesterol metabolism and effectively reduce cardiovascular disease factors.

### Example 5: Fat Decomposition Test

A. Materials and Instruments:

1. Cell line: Mouse bone marrow stromal cells, purchased from ATCC, cell number: CRL-2749, hereinafter referred to as OP9 cells.

2. Cell medium: MEMα (Minimum Essential Medium Alpha Medium) (purchased from Gibco, product number: 12000-014); and 20% of fetal bovine serum (purchased from Gibco, product number: 10437-028) and 1% of penicillin-streptomycin (purchased from Gibco, product number: 15140122) were added.

3. Test kit: Glycerol cell-based assay kit; Purchased from Cayman; product number: 10011725.

4. Detection instrument: ELISA reader, purchased from BioTek (USA).

B. Test Process:

1. The OP9 cells were inoculated into a 24-well culture plate containing 500 μL of cell medium in each well according to a density of 8×10⁴ cells per well, and cultured at 37° C. for 7 d. The cell medium was replaced once every 3 d. Herein, the OP9 cells were divided into two test groups, i.e., a Mock group and an experimental group, respectively. Each group underwent trial in triplicate.

2. After culturing for 7 d, the oil drop formation condition of each group was observed by using a microscope to confirm that the cells of each group were completely differentiated.

3. After observation, the medium of each group was replaced with an experimental medium. The experimental medium of the Mock group was a differentiation medium without the sample, and the experimental medium of the experimental group was a differentiation medium containing 0.125% (v/v) of the Lactobacillus johnsonii TCI369 sample prepared in the Example 2. Then, each group was cultured at 37° C. for 7-10 d, and the corresponding experimental medium of each group was replaced once every 3 d.

4. After culturing, 25 μL of the experimental medium was taken out from each well in each group, and the glycerol secretion content of the differentiated cells of each group was measured by using the glycerol cell-based assay kit. Therefore, after the experimental medium taken from each group was processed according to the test process provided by the glycerol cell-based assay kit, the light absorption value of 540 nm (OD₅₄₀) in each well was measured by the ELISA reader.

C. Test Result:

The relative glycerol secretion content of all groups was calculated according to the following formula: relative glycerol secretion content (%)=(OD₅₄₀ value of each group/OD₅₄₀ value of the Mock group)×100%.

The statistically significant difference between the test results of the Mock group and other groups is obtained by statistical analysis of student t-test. In the figure, “*” represents that the p value is less than 0.05 as compared with the Mock group, “*” represents that the p value is less than 0.01 as compared with the Mock group, and “***” represents that the p value is less than 0.001 as compared with the Mock group.

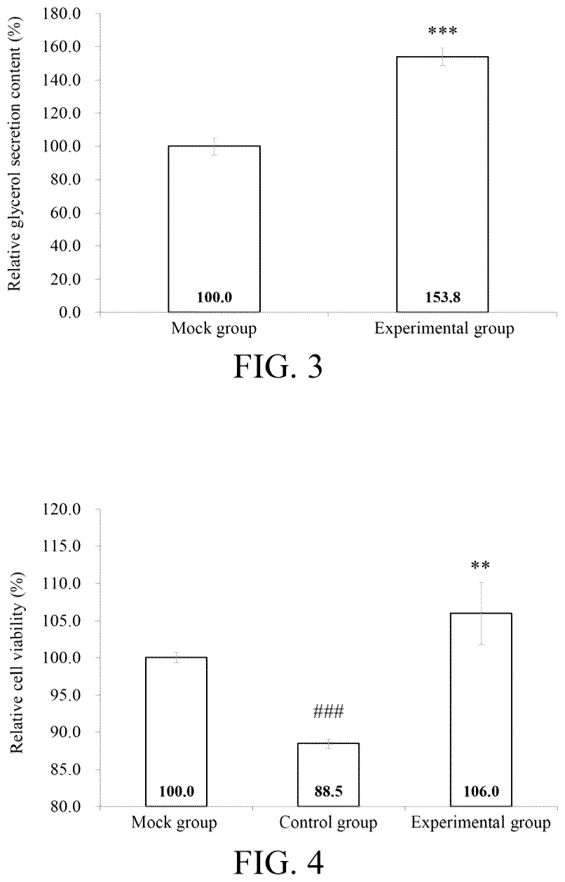

Refer to FIG. 3, the cells in the Mock group are processed without any sample, so the test result of the Mock group represents the performance of the cells under a normal physiological metabolism condition. Here, in a case where a relative glycerol secretion content of the Mock group was set as 100%, and a relative glycerol secretion content of the experimental group was 153.8%. That is, compared with the Mock group, the relative glycerol secretion content of the experimental group was significantly increased by about 53.8% after the Lactobacillus johnsonii TCI369 sample was added into the cells in the experimental group.

Therefore, the Lactobacillus johnsonii TCI369 sample can significantly improve the glycerol secretion content. In other words, it could be seen from the experimental results that the Lactobacillus johnsonii TCI369 and/or metabolites thereof have the effect of significantly promoting fat decomposition. The Lactobacillus johnsonii TCI369 and/or metabolites thereof can improve the lipolysis efficiency, accelerate fat metabolism and effectively reduce cardiovascular disease factors.

### Example 6: UV Resistance and Defense Test

A. Materials and Instruments:

1. Cell line: Human retinal pigmented epithelium, purchased from ATCC, cell number: CRL-2302, hereinafter referred to as ARPE-19 cells.

2. Cell medium: DMEM (Dulbecco's modified Eagle's medium) (purchased from Gibco, product number: 12100-046) and Ham's F12 medium (purchased from Gibco, product number: 21700-026) were mixed at equal amount; and 10% of fetal bovine serum (purchased from Gibco, product number: 10437-028), 0.5 mM of sodium pyruvate (purchased from Gibco, product number: 11360-070) and 15 mM of HEPES buffer (purchased from Gibco, product number 15630-080) were added.

3. 4 mg/mL MTT: Prepared with MTT (purchased from AMERSCO, product number: 0793-5G) and DPBS.

4. DMSO: Purchased from ECHO; Product number: DA1101-000000-72EC.

5. UV radiation box: Purchased from Vilber.

6. Microplate reader (ELISA reader): Purchased from BioTek Company (USA).

B. Test Process:

1. The ARPE-19 cells were inoculated into a 96-well culture plate containing 200 μL of cell medium in each well according to a density of 5×10³ cells per well, and cultured at 37° C. for 24 h. The ARPE-19 cells were divided into three test groups, i.e., a Mock group, a control group and an experiment group, respectively. Each group underwent trial in triplicate.

2. After culturing for 24 h, the medium of each group was replaced with an experiment medium, and each group was continuously cultured at 37° C. for 24 h. The experiment mediums of the Mock group and the control group were cell medium without the sample, and the experiment medium of the experiment group was a cell medium containing (v/v) of the Lactobacillus johnsonii TCI369 sample prepared in the Example 2.

3. After culturing for 24 h, the ARPE-19 cells of the control group and the experiment group were irradiated in an ultraviolet radiation box by using UVB (ultraviolet light with the irradiation energy of 1.5 J/cm 2) for 10 min.

4. 15 μL of 4 mg/mL MTT was added into each group, and cultured at 37° C. for 4 h.

5. After culturing for 4 h, the experiment medium of each group was removed, and 50 μL of DMSO was added into each group to dissolve Formazan crystals. Each group was put on a vibration machine and acted for 10 min.

6. The light absorption value of 570 nm (OD₅₇₀) in each group was measured by the ELISA reader.

C. Test Result:

The relative cell viability of all groups was calculated according to the following formula: relative cell viability (%)=(OD₅₇₀ value of each group/OD₅₇₀ value of the Mock group)×100%.

The statistically significant difference between the test results of the Mock group and other groups and the control group and other groups is obtained by statistical analysis of student t-test. In the figure, “#” represents that the p value is less than 0.05 as compared with the Mock group, “##” represents that the p value is less than 0.01 as compared with the Mock group, and “###” represents that the p value is less than 0.001 as compared with the Mock group. In the figure, “*” represents that the p value is less than 0.05 as compared with the control group, “*” represents that the p value is less than 0.01 as compared with the control group, and “***” represents that the p value is less than 0.001 as compared with the control group.

Refer to FIG. 4, the cells in the Mock group are not stimulated by ultraviolet light and are not processed with the sample, so the test result of the Mock group represents the performance of the ARPE-19 cells under a normal physiological metabolism condition. Here, in a case where a relative cell viability of the Mock group was set as 100%, a relative cell viability of the control group was 88.5%, and a relative cell viability of the experimental group was 106.0%. That is, compared with the Mock group, the relative cell viability of the control group was significantly decreased by about 11.5% after the ARPE-19 cells of the control group are stimulated by the ultraviolet light. Compared with the control group, the relative cell viability of the experimental group was significantly increased by about 19.8% after the ARPE-19 cells of the experimental group are stimulated by the ultraviolet light after the Lactobacillus johnsonii TCI369 sample was added. Compared with the Mock group, the relative cell viability of the experimental group was increased by about 6.0%.

Therefore, the Lactobacillus johnsonii TCI369 sample can significantly improve the retina cell viability reduced by the ultraviolet light. In other words, it could be seen from the experimental results that the Lactobacillus johnsonii TCI369 and/or metabolites thereof have the effect of significantly reducing the damage of the ultraviolet light to the retina cells. The Lactobacillus johnsonii TCI369 and/or metabolites thereof can resist ultraviolet light. The Lactobacillus johnsonii TCI369 and/or metabolites thereof can reduce the damage of the ultraviolet light to eye cells and effectively protect eyes.

### Example 7: Subject Test: Blood Test

A. Test Process:

Ten adult subjects over the age of 20 who were heavily using 3C products or had high blood lipids took one test capsule daily for 4 consecutive weeks (i.e., 28 days). The test capsule included 100 mg of Lactobacillus johnsonii TCI369 viable bacteria (obtained from the Example 2), 4 mg of magnesium stearate, 4 mg of silicon dioxide and 292 mg of indigestible dextrin. In addition, before taking the test capsule (hereinafter referred to as week 14 d after taking the test capsule (hereinafter referred to as week 2) and 28 d after taking the test capsule (hereinafter referred to as week 4), blood samples were drawn from the subjects to detect the changes in the concentrations of triglycerides, high-density lipoproteins (HDL) and very low-density lipoproteins (VLDL) in blood before and after taking the test capsule.

The concentration of triglycerides, HDL and VLDL in blood of the subjects in this example was determined by LEZEN medical laboratory (Taiwan), with reference to the blood test standards announced by the Ministry of Health and Welfare (Taiwan).

B. Test Result:

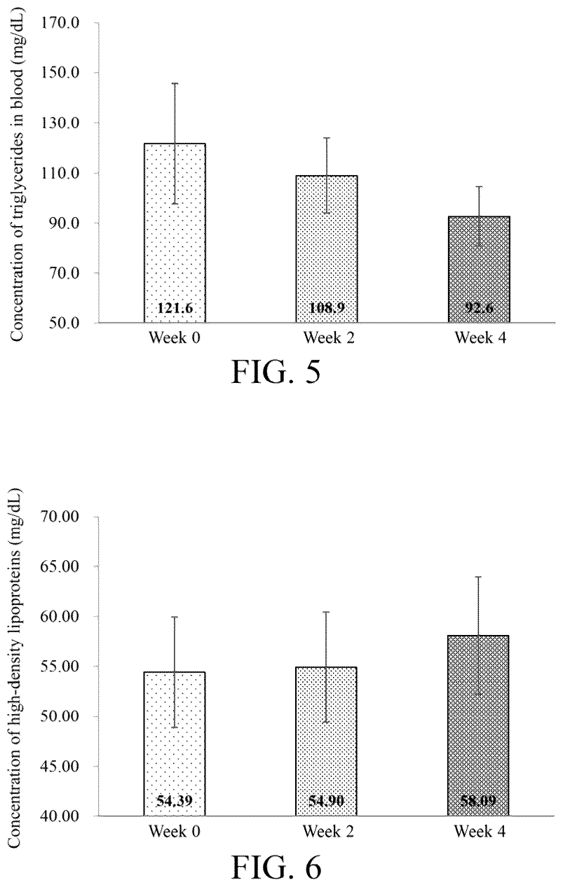

Refer to FIG. 5, FIG. 5 shows the changes in the concentration of triglycerides in blood of the subjects before and after taking the test capsule. The concentration of triglycerides in blood of the subjects at week 0 was about 121.6 mg/dL; the concentration of triglycerides in blood of the subjects at week 2 (i.e., after taking the Lactobacillus johnsonii TCI369 viable bacteria for 2 consecutive weeks) was reduced to about 108.9 mg/dL; and the concentration of triglycerides in blood of the subjects at week 4 (i.e., after taking the Lactobacillus johnsonii TCI369 viable bacteria for 4 consecutive weeks) was reduced to about 92.6 mg/dL. In other words, compared with the condition before taking the test capsule, the concentration of triglycerides in blood of the subjects can be reduced by 10.4% after taking the Lactobacillus johnsonii TCI369 for 2 consecutive weeks. Compared with the condition before taking the test capsule, the concentration of triglycerides in blood of the subjects can be reduced by 23.8% after taking the Lactobacillus johnsonii TCI369 for 4 consecutive weeks. Moreover, the proportion of people improved reached 70%. Therefore, the Lactobacillus johnsonii TCI369 viable bacteria can reduce the concentration of triglycerides in blood. In other words, the Lactobacillus johnsonii TCI369 and/or metabolites thereof have the effect of reducing triglycerides.

Refer to FIG. 6, FIG. 6 shows the changes in the concentration of HDL in blood of the subjects before and after taking the test capsule. The concentration of HDL in blood of the subjects at week 0 was about 54.39 mg/dL; the concentration of HDL in blood of the subjects at week 2 (i.e., after taking the Lactobacillus johnsonii TCI369 viable bacteria for 2 consecutive weeks) was increased to about 54.90 mg/dL; and the concentration of HDL in blood of the subjects at week 4 (i.e., after taking the Lactobacillus johnsonii TCI369 viable bacteria for 4 consecutive weeks) was increased to about 58.09 mg/dL. In other words, compared with the condition before taking the test capsule, the concentration of HDL in blood of the subjects can be increased by 0.9% after taking the Lactobacillus johnsonii TCI369 for 2 consecutive weeks. Compared with the condition before taking the test capsule, the concentration of HDL in blood of the subjects can be increased by 6.8% after taking the Lactobacillus johnsonii TCI369 for 4 consecutive weeks. Moreover, the proportion of people improved reached 70%. Therefore, the Lactobacillus johnsonii TCI369 viable bacteria can increase the concentration of HDL in blood. In other words, the Lactobacillus johnsonii TCI369 and/or metabolites thereof have the effects of increasing HDL and increasing good cholesterol.

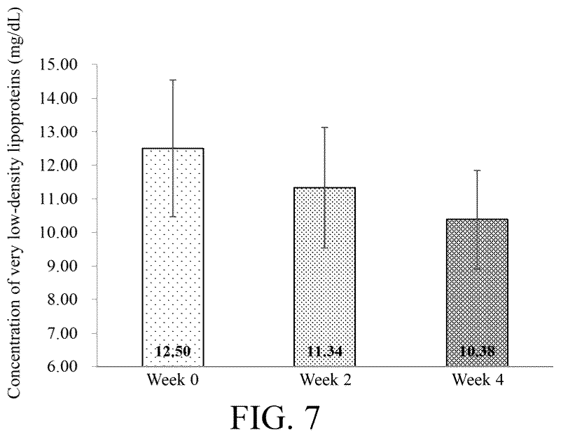

Refer to FIG. 7, FIG. 7 shows the changes in the concentration of VLDL in blood of the subjects before and after taking the test capsule. The concentration of VLDL in blood of the subjects at week 0 was about 12.50 mg/dL; the concentration of VLDL in blood of the subjects at week 2 (i.e., after taking the Lactobacillus johnsonii TCI369 viable bacteria for 2 consecutive weeks) was reduced to about 11.34 mg/dL; and the concentration of VLDL in blood of the subjects at week 4 (i.e., after taking the Lactobacillus johnsonii TCI369 viable bacteria for 4 consecutive weeks) was reduced to about 10.38 mg/dL. In other words, compared with the condition before taking the test capsule, the concentration of VLDL in blood of the subjects can be reduced by 9.3% after taking the Lactobacillus johnsonii TCI369 for 2 consecutive weeks. Compared with the condition before taking the test capsule, the concentration of VLDL in blood of the subjects can be reduced by 17.0% after taking the Lactobacillus johnsonii TCI369 for 4 consecutive weeks. Moreover, the proportion of people improved reached 70%. Therefore, the Lactobacillus johnsonii TCI369 viable bacteria can reduce the concentration of VLDL in blood. In other words, the Lactobacillus johnsonii TCI369 and/or metabolites thereof have the effects of reducing low-density lipoproteins and reducing bad cholesterol.

### Example 8: Subject Test: Microbiota Detection

A. Test Process:

Ten adult subjects over the age of 20 who were heavily using 3C products or had high blood lipids took one test capsule daily for 4 consecutive weeks (i.e., 28 days). The test capsule included 100 mg of Lactobacillus johnsonii TCI369 viable bacteria (obtained from the Example 2), 4 mg of magnesium stearate, 4 mg of silicon dioxide and 292 mg of indigestible dextrin. In addition, before taking the test capsule (hereinafter referred to as week and 28 d after taking the test capsule (hereinafter referred to as week 4), the subjects were subjected to intestinal microbiota detection and gut-associated pathogenic bacterium protein expression level detection. The intestinal microbiota detection and the gut-associated pathogenic bacterium protein expression level detection were carried out in a way that the subjects pasted a clean excrement collection bag on a toilet cover by themselves so as to facilitate subsequent excrement sampling. Before sampling, the subjects would empty urine to avoid polluting excrement samples, then took a proper amount of samples in a sampling tube containing a preservation solution, and then delivered the samples to BIOTOOLS Biotechnology Co., Ltd. for sequencing analysis or comparative analysis.

The comparative analysis was on the basis of a 16S sequencing analysis result, a GreenGenes database and a KEGG orthology copy number relational table, and metabolic pathway functions of three levels of the KEGG database was predicted, and a detection result of the gut-associated pathogenic bacterium protein gene expression was generated. In other words, the increase of the gut-associated pathogenic bacterium protein gene expression detected from the excrement sample represented the increase of the gut-associated pathogenic bacterium protein expression level.

B. Test Result:

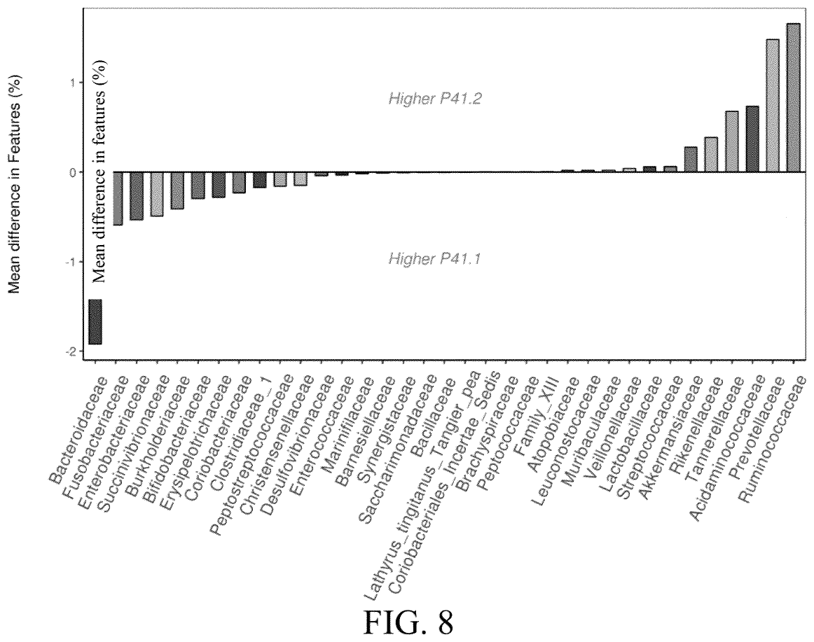

Refer to FIG. 8, FIG. 8 shows the mean difference in features of intestinal microbiota of the subjects before and after taking the test capsule. After taking the Lactobacillus johnsonii TCI369 for 4 consecutive weeks, the abundance of Ruminococcacaceae, Prevotellaceae, Tannerellaceae, Rikenellaceae, Akkermansiaceae, Lactobacillaceae and the like of the intestinal microbiota of the subjects were increased. Also, after taking the Lactobacillus johnsonii TCI369 for 4 consecutive weeks, the abundance of Fusobacteriaceae, Enterobacteriaceae, Erysipelotrichaceae, Clostridiaceae 1, Peptostreptococcaceae, Desulfovibrionaceae, Enterococcaceae and the like of the intestinal microbiota of the subjects were reduced. The strains of the Ruminococcacaceae, Prevotellaceae, Tannerellaceae, Rikenellaceae, Akkermansiaceae and Lactobacillaceae belong to intestinal probiotics, and the strains of the Fusobacteriaceae, Enterobacteriaceae, Erysipelotrichaceae, Clostridiaceae 1, Peptostreptococcaceae, Desulfovibrionaceae and Enterococcaceae belong to intestinal pathogenic bacteria. Therefore, the Lactobacillus johnsonii TCI369 viable bacteria can effectively regulate the balance of the intestinal microbiota. In other words, the Lactobacillus johnsonii TCI369 and/or the metabolites thereof have the effects of increasing the abundance of the intestinal probiotics, reducing the abundance of the intestinal pathogenic bacteria and realizing healthier intestinal tracts.

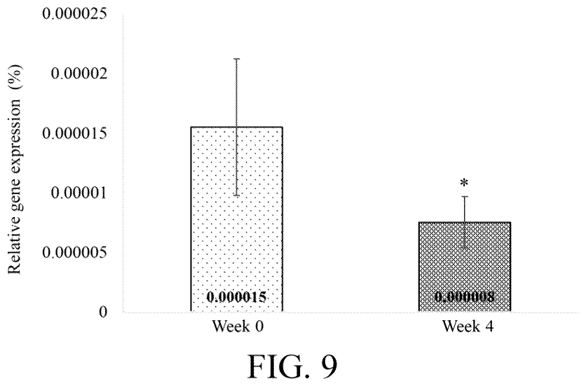

Refer to FIG. 9, FIG. 9 shows the changes in the relative gene expression of gut-associated pathogenic bacteria proteins of the subjects before and after taking the test capsule. The relative gene expression of the gut-associated pathogenic bacteria proteins of the subjects at week 0 was about 0.000015%, and the relative gene expression of the gut-associated pathogenic bacteria proteins of the subjects at week 4 (i.e., after taking the Lactobacillus johnsonii TCI369 viable bacteria for 4 consecutive weeks) was reduced to about 0.000008%. In other words, compared with the condition before taking the test capsule, the relative gene expression of the gut-associated pathogenic bacteria proteins of the subjects can be reduced by 46.7% after taking the Lactobacillus johnsonii TCI369 for 4 consecutive weeks. Therefore, the Lactobacillus johnsonii TCI369 viable bacteria can reduce the relative gene expression of the gut-associated pathogenic bacteria proteins. By detecting the expression level of the gut-associated pathogenic bacteria proteins, the state that pathogenic bacteria in the intestinal tract invade intestinal epithelial cells through KEGG PATHWAY: ko05100 can be evaluated. In other words, the Lactobacillus johnsonii TCI369 and/or metabolites thereof can significantly reduce the gene expression of the gut-associated pathogenic bacteria proteins and reduce the gut-associated pathogenic bacteria proteins expression level. The Lactobacillus johnsonii TCI369 and/or metabolites thereof can significantly reduce the invasion of intestinal pathogenic bacteria into the intestinal epithelial cells and prevent the pathogenic bacteria from invading the intestinal tract. The Lactobacillus johnsonii TCI369 and/or metabolites thereof have the effects of strengthening the intestinal barrier and improving the intestinal barrier function.

In conclusion, the Lactobacillus johnsonii according to any embodiment can increase the level of unsaturation in fatty acids. In some embodiments, the Lactobacillus johnsonii or metabolites thereof according to any embodiment are suitable for preparing the composition for increasing the level of unsaturation in fatty acids. In some embodiments, the method for increasing the level of unsaturation in fatty acids includes: administering to the subject in need thereof the composition including the Lactobacillus johnsonii or metabolites thereof according to any embodiment. In some embodiments, the composition has the function of increasing the level of unsaturation in fatty acids. In some embodiments, the Lactobacillus johnsonii, the metabolites thereof or the prepared composition thereof also has one or more functions of decomposing fat, metabolizing cholesterol, reducing triglycerides, reducing low-density lipoproteins, increasing high-density lipoproteins, regulating the balance of intestinal microbiota, strengthening the intestinal barrier, resisting ultraviolet light and protecting eyes. In some embodiments, the method for decomposing fat, metabolizing cholesterol, reducing triglycerides, reducing low-density lipoproteins, increasing high-density lipoproteins, regulating the balance of intestinal microbiota, strengthening the intestinal barrier, resisting ultraviolet light and/or protecting eyes includes: administering to the subject in need thereof the Lactobacillus johnsonii, the metabolites thereof or the prepared composition thereof.

What is claimed is:

1. Lactobacillus johnsonii, wherein the Lactobacillus johnsonii is Lactobacillus johnsonii TCI369 with an accession number of DSM 34008.

2. A method for increasing the level of unsaturation in fatty acids, comprising: administering to a subject in need thereof a composition comprising Lactobacillus johnsonii or metabolites thereof, wherein the Lactobacillus johnsonii is Lactobacillus johnsonii TCI369 with an accession number of DSM 34008.

3. The method according to claim 2, wherein the Lactobacillus johnsonii is used for decomposing fat or metabolizing cholesterol of the subject.

4. The method according to claim 2, wherein the Lactobacillus johnsonii is used for reducing triglycerides of the subject.

5. The method according to claim 2, wherein the Lactobacillus johnsonii is used for reducing low-density lipoproteins of the subject.

6. The method according to claim 2, wherein the Lactobacillus johnsonii is used for increasing high-density lipoproteins of the subject.

7. The method according to claim 2, wherein the Lactobacillus johnsonii is used for regulating the balance of intestinal microbiota of the subject.

8. The method according to claim 2, wherein the Lactobacillus johnsonii is used for strengthening intestinal barrier of the subject.

9. The method according to claim 2, wherein the Lactobacillus johnsonii is used for resisting ultraviolet light for the subject.

10. The method according to claim 9, wherein the Lactobacillus johnsonii is used for protecting eyes of the subject.
